(19) United States
(12) Patent Application Publication
(10) Pub. No.: US 2023/0293752 A1
(43) Pub. Date: Sep. 21, 2023

(54) CHEMICAL SPRAYING SYSTEM AND METHOD FOR OPERATING SAME

(71) Applicant: Panasonic Intellectual Property Management Co., Ltd., Osaka (JP)

(72) Inventor: Kota KIMURA, Saitama (JP)

(21) Appl. No.: 18/018,319

(22) PCT Filed: Oct. 11, 2021
(86) PCT No.: PCT/JP2021/037603

§ 371 Date: Jan. 27, 2023

(30) Foreign Application Priority Data
Oct. 13, 2020 (JP) 2020-172676

(51) Int. Cl. A61L 2/22; B05B 7/26; B05B 7/04

(52) U.S. Cl. CPC A61L 2/22; B05B 7/267; B05B 7/0416; A61L 2202/15; A61L 2202/14

(57) ABSTRACT

A control unit performs control such that, in a state where water does not flow into a water path, an on-off valve is opened to supply air from a second air path to the water path and a mixed solution path to discharge residual water and thereafter supply of the air is stopped.

The present disclosure relates to a chemical spraying system that sprays a mixed solution of a sanitizing solution and water as a chemical for sanitization or sterilization, and a method for operating the same.

### BACKGROUND ART

A system for spraying a chemical is used for sanitization or sterilization.

For example, PTL 1 discloses a pH controlling apparatus that stably controls a pH value of sterilized water generated by mixing carbon dioxide gas with raw water.
PTL 1: JP 2010-115639 A

### SUMMARY OF INVENTION

In the conventional pH controlling apparatus described above, it is not assumed that bacteria are generated in the apparatus when operation thereof is stopped. In a configuration in which a sanitizing solution and water are mixed, bacteria may propagate in water remaining in a water path that supplies water.

Therefore, an object of the chemical spraying system and the method for operating the same of the present disclosure is to suppress propagation of bacteria in residual water in a path through which water passes.

According to one aspect of the present disclosure, there is provided a chemical spraying system including: at least one two-fluid nozzle to which a gas and a liquid are supplied and through which the gas and the liquid are sprayed; a water supply unit that supplies water as the liquid to a liquid-side inlet of the at least one two-fluid nozzle; a sanitizing solution supply unit that supplies a sanitizing solution as the liquid to the liquid-side inlet of the at least one two-fluid nozzle; an air supply unit that supplies air as the gas to a gas-side inlet of the at least one two-fluid nozzle; a liquid mixing unit that mixes the water and the sanitizing solution to generate a mixed solution of the water and the sanitizing solution; a water path that supplies the water from the water supply unit to the liquid mixing unit; a sanitizing solution path that supplies the sanitizing solution from the sanitizing solution supply unit to the liquid mixing unit; a mixed solution path that supplies the mixed solution from the liquid mixing unit to the liquid-side inlet of the at least one two-fluid nozzle; a first air path that supplies the air from the air supply unit to the gas-side inlet of the at least one two-fluid nozzle; a second air path that supplies the air from the air supply unit to the water path; an on-off valve that opens and closes the second air path; and a control unit that performs control such that, in a state where the water does not flow into the water path, the on-off valve is opened to supply the air from the second air path to the water path and the mixed solution path and thereafter supply of the air is topped.

According to another aspect of the present disclosure, there is provided a method for operating a chemical spraying system including at least one two-fluid nozzle to which a gas and a liquid are supplied and through which the gas and the liquid are sprayed, a water supply unit that supplies water as the liquid to a liquid-side inlet of the at least one two-fluid nozzle, a sanitizing solution supply unit that supplies a sanitizing solution as the liquid to the liquid-side inlet of the at least one two-fluid nozzle, an air supply unit that supplies air as the gas to a gas-side inlet of the at least one two-fluid nozzle, a liquid mixing unit that mixes the water and the sanitizing solution to generate a mixed solution of the water and the sanitizing solution, a water path that supplies the water from the water supply unit to the liquid mixing unit, a sanitizing solution path that supplies the sanitizing solution from the sanitizing solution supply unit to the liquid mixing unit, a mixed solution path that supplies the mixed solution from the liquid mixing unit to the liquid-side inlet of the at least one two-fluid nozzle, a first air path that supplies the air from the air supply unit to the gas-side inlet of the at least one two-fluid nozzle, a second air path that supplies the air from the air supply unit to the water path, an on-off valve that opens and closes the second air path, and a control unit that performs control such that, in a state where the water does not flow into the water path, the on-off valve is opened to supply the air from the second air path to the water path and the mixed solution path and thereafter supply of the air is stopped, the method including: by the control unit, opening the on-off valve and supplying the air to the water path and the mixed solution path from the air supply unit via the second air path in a state where the water supply unit does not cause the water to flow into the water path; and thereafter by the control unit, performing control such that supply of the air to the water path is stopped.

### Advantageous Effects of Invention

According to the aspects of the present disclosure, since control is performed such that the on-off valve is opened to supply the air from the second air path to the water path in a state where the water does not flow into the water path, and thereafter, supply of the air is stopped, residual water in the path through which the water passes can be caused to flow out with the supplied air. As a result, it is possible to suppress propagation of bacteria in the residual water in the path through which the water passes. In addition, since the liquid in the mixing path can also be discharged, it is possible to suppress bacteria from propagating in the residual water in the mixing path regardless of the concentration of the sanitizing solution.

### BRIEF DESCRIPTION OF DRAWINGS

FIG. 1 is a diagram of a chemical spraying system according to a first embodiment of the present disclosure.

FIG. 2 is a partially enlarged view of a mixing tank in FIG. 1.

FIG. 3 is a diagram of a chemical spraying system according to a second embodiment of the present disclosure.

### DESCRIPTION OF EMBODIMENTS

Hereinafter, embodiments of the present disclosure will be described in detail with reference to the drawings.

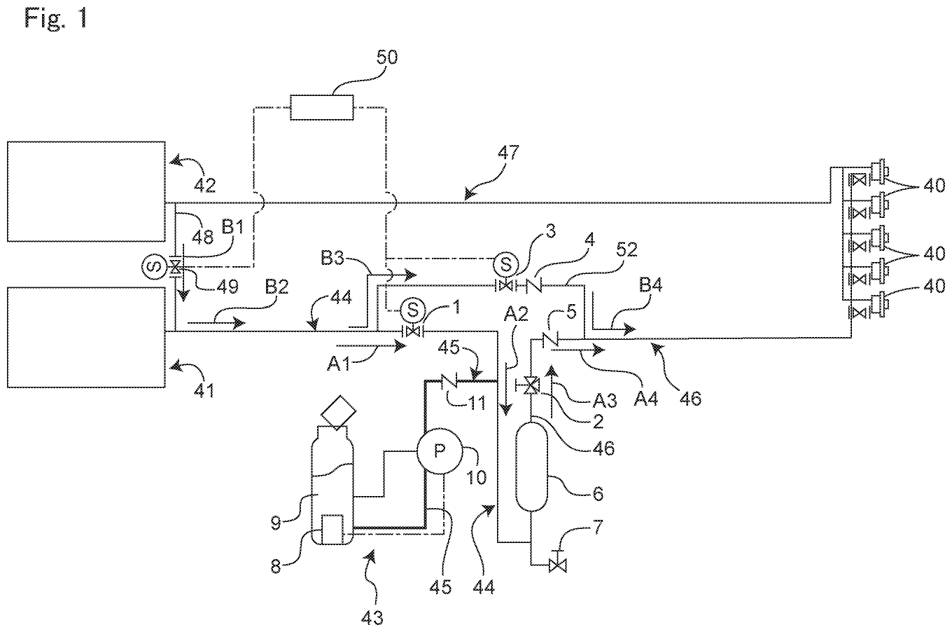

As illustrated in FIG. 1, a chemical spraying system according to a first embodiment of the present disclosure at least includes at least one two-fluid nozzle 40, a water supply unit 41, a sanitizing solution supply unit 43, an air supply unit 42, a mixing tank 6 as an example of a liquid mixing unit, a water path 44, a sanitizing solution path 45, a mixed solution path 46, a first air path 47, a second air path 48, an on-off valve 49, and a control unit 50.

In FIG. 1, a plurality of, for example, five two-fluid nozzles 40 are provided. A downstream end of the first air path 47 and a downstream end of the mixed solution path 46 are connected to each of the two-fluid nozzles 40, a gas and a liquid are supplied to each of the two-fluid nozzles 40 via the first air path 47 and the mixed solution path 46, and each of the two-fluid nozzles sprays the gas and the liquid in a mist form. An example of the gas is air, and an example of the liquid is a mixed solution of water and a sanitizing solution.

The water supply unit 41 is connected to an upstream end of the water path 44 to supply water from a water tank by a pump or from a water pipe to a liquid-side inlet of the two-fluid nozzle via the water path. 44 and the mixed solution path 46.

The sanitizing solution supply unit 43 supplies the sanitizing solution to the liquid-side inlet of the two-fluid nozzle 40. Specifically, as described later, the sanitizing solution is mixed with water to be a mixed solution, and then the mixed solution is supplied to the liquid-side inlet of the two-fluid nozzle 40. As an example, the sanitizing solution supply unit 43 includes a sanitizing solution tank 9 in which the sanitizing solution is held, and a sanitizing solution pump 10 that supplies the sanitizing solution in the sanitizing solution tank 9 to the mixed solution path 46.

The air supply unit 42 is connected to an upstream end of the first air path 47 and supplies air to a gas-side inlet of the two-fluid nozzle 40 via the first air path 47.

The water path 44 is connected to an inflow-port side of the mixing tank 6, and the mixed solution path 46 is connected to an outflow-port side of the mixing tank 6. In the mixing tank 6, water flowing from the water path 44 and the sanitizing solution are mixed to generate a mixed solution of the water and the sanitizing solution, and the mixed solution is caused to flow out through the mixed solution path 46. In FIG. 1, the mixing tank 6 is disposed along the vertical direction, and an inflow port thereof is disposed at a lower end thereof and an outflow port thereof is disposed at an upper end thereof. That is, the inflow port is disposed below the outflow port in the direction of gravity.

The water path 44 is a path that supplies water from the water supply unit 41 to the mixing tank 6. A sanitizing solution supply solenoid valve 1 is disposed in the water path 44. The sanitizing solution supply solenoid valve 1 is disposed between the water supply unit 41 and the connection portion between the sanitizing solution path. 45 and the water path 44 in the water path 44, and is, for example, a normally-closed valve. The sanitizing solution supply solenoid valve 1 functions as a valve for preventing mixture of air by being opened at the time of supplying water and being closed at the time of stopping supply of water.

The sanitizing solution path 45 is a path that is connected to the sanitizing solution supply unit 43 and a portion between the water supply unit 41 and the mixing tank 6 in the water path 44, and supplies the sanitizing solution from the sanitizing solution supply unit 43 to the mixing tank 6 via part of the water path 44. Specifically, the sanitizing solution path 45 connects the tank 9 and the portion between the water supply unit 41 and the mixing tank 6 in the water path 44 via the sanitizing solution pump 10.

The mixed solution path 46 is a path that connects the mixing tank 6 and the liquid-side inlet of the two-fluid nozzle 40, and supplies the mixed solution from the mixing tank 6 to the liquid-side inlet of the two-fluid nozzle 40.

The first air path 47 is a path that supplies air from the air supply unit 42 to the gas-side inlet of the two-fluid nozzle 40.

The second air path 48 is a path that connects the vicinity of the upstream end of the first air path 47 and the vicinity of the upstream end of the water path 44 and supplies air from the air supply unit 42 to the water path 44.

The on-off valve 49 is disposed in the second air path 48 and opens and closes the second air path 48.

In a state where water does not flow into the water path 44, the control unit 50 performs control such that the on-off valve 49 is opened to supply air from the second air path 48 to the water path 44 and the mixed solution path 46, and thereafter supply of the air is stopped. The air supplied to the second air path 48 enters the water path 44 and enters a bypass path 52 as described later. The control unit 50 controls opening and closing of the on-off valve 49, and also controls supply from the water supply unit 41 such that a state where water does not flow into the water path 44 is achieved. Here, the state where water does not flow into means not stop of water supply, but, for example, a state where a pressure of water supply is made lower than a pressure of air supply so that water does not flow into the water path 44, instead of water supply stop.

Furthermore, the control unit 50 can perform a desired operation by controlling operations of the water supply unit 41, the sanitizing solution supply unit 43, the air supply unit 42, and the on-off valve 49.

The chemical spraying system may further include the bypass path 52 and a switching mechanism.

That is, the bypass path 52 is connected, from the water path 44, to the mixed solution path 46 without passing through the mixing tank 6.

As an example, the switching mechanism that switches between the bypass path 52 and the water path 44 includes a bypass solenoid valve which is an on-off valve, and the sanitizing solution supply solenoid valve 1, and operations of them are controlled by the control unit 50. The switching mechanism only needs to have a function of switching to either the bypass path 52 or the water path 44, and an example thereof is a three-way valve or an on-off valve on the path. Here, upon switching to the bypass path 52, the bypass solenoid valve 3 is opened and the sanitizing solution supply solenoid valve 1 is closed. Upon switching to the water path 44, the bypass solenoid valve 3 is closed and the sanitizing solution supply solenoid valve 1 is opened. Switching can be controlled by the control unit 50.

The control unit 50 supplies the sanitizing solution to the mixing tank 6, opens the on-off valve 49 after stopping water supply to the water path 44, switches the water path 44 to the bypass path 52 by the bypass solenoid valve 3 and the sanitizing solution supply solenoid valve 1 of the switching mechanism, supplies air to the bypass path 52 via the second air path 48, and stops supply of the air.

As a result, at least the liquid remaining in the water path 44 on the upstream side of the connection portion between the water path 44 and the bypass path 52 is discharged to the nozzle side y the supplied air, and propagation of bacteria in the remaining liquid can be suppressed. Furthermore, also the liquid remaining in the mixed solution path 46 on the downstream side of the connection portion between the bypass path 52 and the mixed solution path 46 is discharged to the nozzle side by the supplied air, and propagation of bacteria in the remaining liquid can be suppressed regardless of the concentration of the sanitizing solution.

The chemical spraying system may further include the following configuration, and the chemical spraying system can more smoothly perform a spraying operation by including the following configuration.

A three-way valve 2 is disposed in the mixed solution path 46 on the outflow-port side of the mixing tank 6 and functions as a valve for measuring the concentration of spray water.

A check valve 4 is disposed on the downstream side of the bypass solenoid valve 3 of the bypass path 52 and is a valve for preventing backflow during normal driving.

A check valve 5 is disposed on the downstream side of the three-way valve 2 in the mixed solution path 46, and is a valve for preventing backflow of air at the time of stop.

A drain valve 7 is a valve for draining that is connected to the inflow-port side on the lower part of the mixing tank 6 and discharges the liquid in the mixing tank 6.

A float sensor 8 is a sensor that is disposed in a lower portion of the sanitizing solution tank 9 disposed along the vertical direction and detects the remaining amount of the sanitizing solution in the sanitizing solution tank 9. A detection result of the float sensor 8 is input to the sanitizing solution pump 10 and driving of the sanitizing solution pump 10 is stopped when the detection result becomes a predetermined remaining amount or less.

A check valve 11 is disposed in the sanitizing solution path 45 and is a valve for preventing backflow of water from the water path 44.

The mixing tank 6 will be described below.

The sanitizing solution pump 10 is a metering pump, and intermittently supplies the sanitizing solution to the sanitizing solution path 45 and thus to the water path 44. Then, the concentration of the sanitizing solution becomes non-uniform in the water path 44. In order to prevent this, the mixing tank 6 is provided between the sanitizing solution pump 10 and the nozzle 40 to alleviate concentration fluctuation of the sanitizing solution.

That in a case where the sanitizing solution is injected into the water path 44 by using a diaphragm metering pump as an example of the sanitizing solution pump 10, the sanitizing solution is intermittently injected into the water path 44 through which water flows due to the characteristics of the sanitizing solution pump 10. Therefore, there has been an issue that the concentration of the sanitizing solution varies on a pipe path of the water path 44.

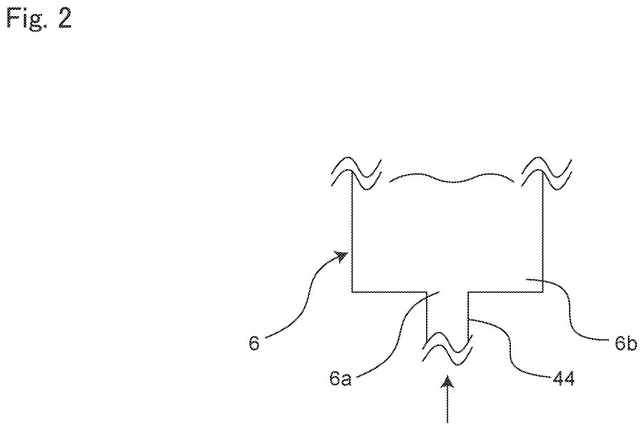

Therefore, in order to solve this issue, as illustrated in FIG. 2, a pipe or a tank having an inner diameter larger than at least the pipe inner diameter of the water path 44, that is, the mixing tank 6 is provided on the downstream side of the connection portion between the sanitizing solution path 45 from the sanitizing solution pump 10 and the water path 44.

With such a configuration, when a diluted liquid having a non-uniform concentration distribution flows into the mixing tank 6 having an inner diameter larger than the inner diameter of the water path 44, a flow path rapidly expands in the mixing tank 6. At this time, the inflow fluid is diffused by a negative pressure space 6b formed in an outer peripheral portion in the vicinity of an inflow port 6a of the mixing tank 6 whose inner diameter is larger than the inner diameter of the water path 44, That is, since the vicinity of the inflow port of the mixing tank 6 is bent in an L shape instead of a gentle inclined surface, the negative pressure space 6b is created at an edge in the vicinity of the inflow port of the mixing tank 6 due to a sharp change from the pipe inner diameter of the water path 44 to the tank inner diameter, and flow of the fluid is disturbed and the liquid is diffused. In addition, since the inner diameter of the mixing tank 6 is large, in-pipe flow velocity decreases, and a spot where a portion with high concentration and a portion with low concentration are mixed is generated. Due to these actions, the concentration of the sanitizing solution is made uniform in the mixing tank 6. As a result, the above-described issue can be solved, and the chemical concentration of a mist sprayed from the nozzle 40 can be made constant.

Here, larger than the pipe inner diameter of the water path 44 means that for example, the mixing tank 6 having an inner diameter of about two to ten times the pipe inner diameter of the water path 44 is practically preferable. This is because there is a possibility that mixing of water and the sanitizing solution is not sufficient in the case of being less than twice the pipe inner diameter of the water path 44. This is also because the mixing tank 6 becomes excessively large if the inner diameter of the mixing tank 6 exceeds ten times the pipe inner diameter of the water path 44.

According to the above-described configuration, operation is performed as follows by operation control by the control unit 50.

First, a spraying operation will be described.

In a state where the on-off valve 49 is closed, air is supplied from the air supply unit 42 to the gas-side inlet of the two-fluid nozzle 40 via the first air path 47.

At the same time, water is supplied from the water supply unit 41 to the aid-side inlet of the two-fluid nozzle 40. At this time, water is supplied from the water supply unit 41 to the water path 44 (see arrows A1 and A2 in FIG. 1), passes through the mixing tank 6, and is supplied from the mixed solution path 46 to the liquid-side inlet of the two-fluid nozzle.

Furthermore, at, the same time, the sanitizing solution is supplied from the sanitizing solution tank 9 to the water path 44 via the sanitizing solution path 45 by driving the sanitizing solution pump 10, and then water and the sanitizing solution are mixed in the mixing tank 6 to generate the mixed solution. Thereafter, the mixed solution is supplied to the liquid-side inlet of the two-fluid nozzle 40 via the mixed solution path 46 (see arrows A3 and A4 in FIG. 1).

In each two-fluid nozzle 40, air and the mixed solution form a mist and the mist is sprayed.

Next, operation of discharging the residual water will be described.

First, water supply from the water supply unit 41 is stopped. Instead of stopping the water supply, the pressure of the water supply may be made lower than the pressure of air supply so that water does not flow into the water path 44.

Next, the on-off valve 49 is opened, and the water path 44 is switched to the bypass path 52 by the bypass solenoid valve 3 and the sanitizing solution supply solenoid valve 1 of the switching mechanism. As a result, air is supplied from the air supply unit 42 to the bypass path 52 via the second air path 48 and part of the water path 44 (see arrows B1, B2, and B3 in FIG. 1). The air supplied to the bypass path 52 enters the mixed solution path 46 (see arrow B4 in FIG. 1), and is sprayed or discharged from the nozzle 40. As a result, the residual water that existed in the path through which the air flowed is discharged from the path. Thereafter, supply of air from the air supply unit 42 is stopped.

According to the first embodiment, at the time of stopping the water supply, the residual water that existed in the path through which the air flowed and the water passes, for example, the liquid remaining in the water path 44 on the upstream side of the connection portion between the water path 44 and the bypass path 52 is discharged to the nozzle side by the supplied air, and propagation of bacteria in the residual liquid can be suppressed. Furthermore, also the liquid remaining in the mixed solution path 46 on the downstream side of the connection portion between the bypass path 52 and the mixed solution path 46 is discharged to the nozzle side by the supplied air, and propagation of bacteria in the remaining liquid can be suppressed regardless of the concentration of the sanitizing solution. In addition, the path volume of the path not passing through the bypass path 52 (main path), that is, the path from input of the sanitizing solution to the connection portion between the mixed solution path 46 and the bypass path 52 through the mixing tank 6 is larger than the path volume of the bypass path 52. Therefore, there is an effect that the path passing through the bypass path 52 wastes less liquid when the liquid in the path is discharged by the air supply at the time of stopping the water supply. In addition, since the flow rate of spray from the nozzle 50 is substantially constant, if the path volume of the path through which the air passes when the water supply is stopped is small, discharge time of the liquid in the path can be shortened. Therefore, if the pipe inner diameter of the bypass path. 52 is made smaller than the pipe inner diameter of the main path, the time required for liquid discharge can be reduced.

In addition, since the mixing tank 6 is disposed along the vertical direction and the outflow port is disposed at the upper end thereof, air is less likely to remain in the mixing tank 6 from which air is more easily extracted, and it is possible to make it harder for bacteria to propagate.

Note that the present disclosure is not limited to the above-described embodiment, and can be implemented in various other aspects.

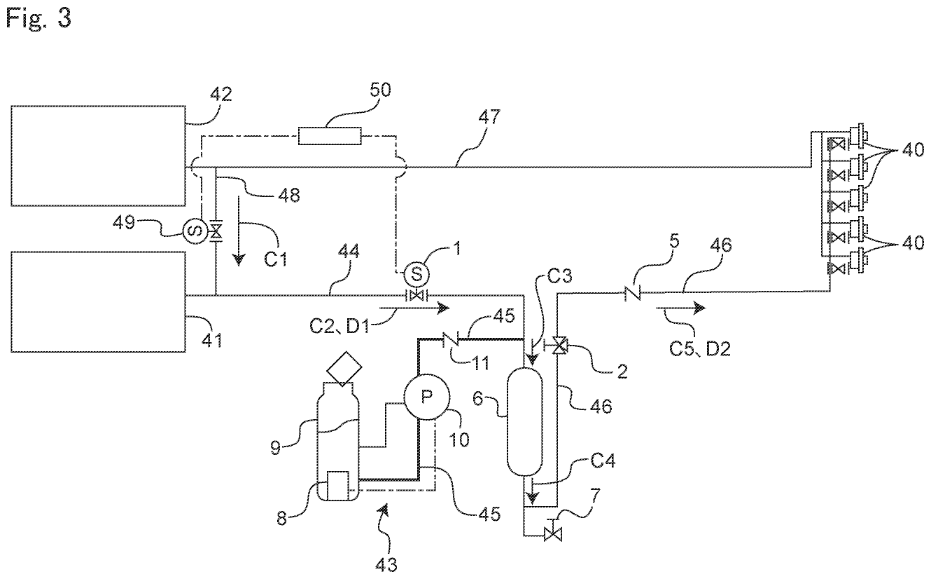

For example, as illustrated in FIG. 3, a chemical spraying system according to a second embodiment is different from that according to the first embodiment in that there is no bypass path 52, an inflow port of a mixing tank 6 disposed along the vertical direction is disposed at an upper end thereof, an outflow port of the mixing tank 6 is disposed at a lower end thereof, and residual water in the mixing tank 6 can be discharged. That is, the inflow port of the mixing tank 6 is disposed above the outflow port in the direction of gravity.

Here, the length of the mixing tank 6 is practically preferably, for example, twice or more and ten times or less the pipe inner diameter of a water path 44. This is because there is a possibility that mixing of water and a sanitizing solution is not sufficient if the length of the mixing tank 6 is less than twice the pipe inner diameter of the water path 44. In addition, the length of the mixing tank 6 exceeds ten times the pipe inner diameter of the water path 44, it takes too much time to supply air to remove the residual water.

According to the above-described configuration, operation is performed as follows by operation control by the control unit 50.

First, a spraying operation will be described.

In a state where the on-off valve 49 is closed, air is supplied from the air supply unit 42 to the gas-side inlet of the two-fluid nozzle 40 via the first air path 47.

At the same time, water supplied from the water supply unit 41 to the liquid-side inlet of the two-fluid nozzle 40. At this time, the water is supplied from the water supply unit 41 to the water path 44 (see arrow D1 in FIG. 3), passes through the mixing tank 6, and is supplied from a mixed solution path 46 to the liquid-side Inlet of the two-fluid nozzle (see arrow D2 in FIG. 3).

Furthermore, at the same time, the sanitizing solution is supplied from the sanitizing solution tank 9 to the water path 44 via the sanitizing solution path 45 by driving the sanitizing solution pump 10, and then water and the sanitizing solution are mixed in the mixing tank 6 to generate the mixed solution. Thereafter, the mixed solution is supplied to the liquid-side inlet of the two-fluid nozzle 40 via the mixed solution path 46 (see arrow D2 in FIG. 3).

In each two-fluid nozzle 40, air and the mixed solution form a mist and the mist is sprayed.

Next, operation of discharging the residual water will be described.

First, water supply from the water supply unit 41 is stopped. Instead of stopping the water supply, the pressure of the water supply may be made lower than the pressure of air supply so that water does not flow into the water path 44.

Next, the on-off valve 49 is opened, and air is supplied from the air supply unit 42 to the mixing tank 6 through the inflow port at the upper end via the second air path 48 and the water path 44 (see arrows C1, C2, and C3 in FIG. 3). The air supplied to the mixing tank. 6 is discharged from the outflow port at the lower end of the mixing tank 6 and enters the mixed solution path 46 (see arrow C4 in FIG. 3) Thereafter, the air reaches the nozzle 40 from the mixed solution path 46 (see arrow C5 in FIG. 3), and is sprayed or discharged from the nozzle 40. As a result, the residual water that existed in the paths through which the air flowed and in the mixing tank 6 is discharged from the paths and the mixing tank 6. Thereafter, supply of air from the air supply unit 42 is stopped.

According to the second embodiment, a residual liquid that existed in the paths 44 and 46 through which the air flowed and the water passes and in the mixing tank 6 is discharged to the nozzle side by the supplied air, and propagation of bacteria in the residual liquid can be suppressed.

Note that in each of the embodiments, the sanitizing solution supply solenoid valve 1 may be disposed on the downstream side of the check valve 5 on the downstream side of the mixing tank 6 instead of being disposed on the upstream side of the mixing tank 6.

Note that by appropriately combining arbitrary embodiments or modified examples among the various embodiments or modified examples described above, the effects of the respective embodiments or modified examples can be achieved. In addition, combinations of embodiments, combinations of working examples, or combinations of an embodiment and a working example are possible, and combinations of features in different embodiments or wording examples are also possible.

Although the present disclosure has been fully described in connection with preferred embodiments with reference to the accompanying drawings, various changes and modifications will be apparent to those skilled in the art. Such changes and modifications are to be understood as being included within the scope of the present disclosure as set forth in the appended claims.

### INDUSTRIAL APPLICABILITY

The chemical spraining system and the method for operating the same according to the present disclosure can prevent bacteria from propagating in residual water in the paths through which water passes, and are useful for spraying a mixed solution of a sanitizing solution and water as a chemical for sanitization or sterilization.

### REFERENCE SIGNS LIST

1. sanitizing solution supply solenoid valve
2. three-way valve
3. bypass solenoid valve
4. check valve
5. check valve
6. mixing tank
6a. inflow port
6h. negative pressure space
7. drain valve
8. float sensor
9. sanitizing solution tank
10. sanitizing solution pump
11. check valve
40. two-fluid nozzle
41. water supply unit
42. air supply unit
43. sanitizing solution supply unit
44. water path
45. sanitizing solution path
46. mixed solution path
47. first air path
48. second air path
49. on-off valve
90. control unit
A1, A2, A3, A4. liquid flow during liquid supply
B1, B2, B3, B4. air flow when liquid supply is stopped
C1, C2, C3, C4, C5. air flow when liquid supply is stopped
D1, D2. liquid flow during liquid supply

1. A chemical spraying system comprising:
at least one two-fluid nozzle to which a gas and a liquid are supplied and through which the gas and the liquid are sprayed;
a water supply unit that supplies water as the liquid to a liquid-side inlet of the at least one two-fluid nozzle;
a sanitizing solution supply unit that supplies a sanitizing solution as the liquid to the liquid-side inlet of the at least one two-fluid nozzle;
an air supply unit that supplies air as the gas to a gas-side inlet of the at least one two-fluid nozzle;
a liquid mixing unit that mixes the water and the sanitizing solution to generate a mixed solution of the water and the sanitizing solution;
a water path that supplies the water from the water supply unit to the liquid mixing unit;
a sanitizing solution path that supplies the sanitizing solution from the sanitizing solution supply unit to the liquid mixing unit;
a mixed solution path that supplies the mixed solution from the liquid mixing unit to the liquid-side inlet of the at least one two-fluid nozzle;
a first air path that supplies the air from the air supply unit to the gas-side inlet of the at least one two-fluid nozzle;
a second air path that supplies the air from the air supply unit to the water path;
an on-off valve that opens and closes the second air path; and
a control unit that performs control such that, in a state where the water does not flow into the water path, the on-off valve is opened to supply the air from the second air path to the water path and the mixed solution path and thereafter supply of the air is stopped.

2. The chemical spraying system according to claim 1, wherein an inflow port of the liquid mixing unit is disposed above an outflow port of the liquid mixing unit in a direction of gravity.

3. The chemical spraying system according to claim 1, wherein an inflow port of the liquid mixing unit is disposed below an outflow port of the liquid mixing unit in a direction of gravity.

4. The chemical spraying system according to claim 1, further comprising:
a bypass path that is connected, from the water path, to the mixing solution path without passing through the liquid mixing unit; and
a switching mechanism that switches between the bypass path and the water path,
wherein the control unit supplies the sanitizing solution to the liquid mixing unit, and, in a state where the water does not flow into the water path, opens the on-off valve and causes the switching mechanism to switch over from the water path to the bypass path to supply the air to the mixing solution path via the second air path, the water path, and the bypass path but not via the liquid mixing unit, and stops supply of the air.

5. The chemical spraying system according to claim 1, wherein the liquid mixing unit is a pipe or a mixing tank having an inner diameter larger than a pipe inner diameter of the water path connected to the inflow port of the liquid mixing unit.

6. A method for operating a chemical spraying system including
at least one two-fluid nozzle to which a gas and a liquid are supplied and through which the gas and the liquid are sprayed,
a water supply unit that supplies water as the liquid to a liquid-side inlet of the at least one two-fluid nozzle,
a sanitizing solution supply unit that supplies a sanitizing solution as the liquid to the liquid-side inlet of the at least one two-fluid nozzle,
an air supply unit that supplies air as the gas to a gas-side inlet of the at least one two-fluid nozzle,
a liquid mixing unit that mixes the water and the sanitizing solution to generate a mixed solution of the water and the sanitizing solution,
a water path that supplies the water from the water supply unit to the liquid mixing unit,
a sanitizing solution path that supplies the sanitizing solution from the sanitizing solution supply unit to the liquid mixing unit,
a mixed solution path that supplies the mixed solution from the liquid mixing unit to the liquid-side inlet of the at least one two-fluid nozzle,
a first air path that supplies the air from the air supply unit to the gas-side inlet of the at least one two-fluid nozzle,
a second air path that supplies the air from the air supply unit to the water path,
an on-off valve that opens and closes the second air path, and
a control unit that performs control such that, in a state where the water does not flow into the water path, the on-off valve is opened to supply the air from the second air path to the water path and the mixed solution path and thereafter supply of the air is stopped,
the method comprising:
by the control unit, opening the on-off valve and supplying the air to the water path and the mixed solution path from the air supply unit via the second air path in a state where the water supply unit does not cause the water to flow into the water path; and thereafter
by the control unit, controlling the supply of the air to the water path to be stopped.

7. The method for operating the chemical spraying system according claim 6, the chemical spraying system further including
a bypass path that is connected, from the water path, to the mixing solution path without passing through the liquid mixing unit, and
a switching mechanism that switches between the bypass path and the water path,
the method further comprising:
by the control unit, supplying the sanitizing solution to the liquid mixing unit;
by the control unit, opening the on-off valve and causing the switching mechanism to switch over from the water path to the bypass path to supply the air from the air supply unit to the mixing solution path via the second air path, the water path, and the bypass path but not via the liquid mixing unit in a state where the water does not flow from the water supply unit into the water path; and thereafter
by the control unit, stopping the supply of the air to the water path.

8. The chemical spraying system according to claim 3 further comprising:
a bypass path that is connected, from the water path, to the mixing solution path without passing through the liquid mixing unit; and
a switching mechanism that switches between the bypass path and the water path,
wherein the control unit supplies the sanitizing solution to the liquid mixing unit, and, in a state where the water does not flow into the water path, opens the on-off valve and causes the switching mechanism to switch over from the water path to the bypass path to supply the air to the mixing solution path via the second air path, the water path, and the bypass path but not via the liquid mixing unit, and stops supply of the air.

9. The chemical spraying system according to claim 2, wherein the liquid mixing unit is a pipe or a mixing tank having an inner diameter larger than a pipe inner diameter of the water path connected to the inflow port of the liquid mixing unit.

10. The chemical spraying system according to claim 3, wherein the liquid mixing unit is a pipe or a mixing tank having an inner diameter larger than a pipe inner diameter of the water path connected to the inflow port of the liquid mixing unit.

11. The chemical spraying system according to claim 4, wherein the liquid mixing unit is a pipe or a mixing tank having an inner diameter larger than a pipe inner diameter of the water path connected to the inflow port of the liquid mixing unit.

12. The chemical spraying system according to claim 8, wherein the liquid mixing unit is a pipe or a mixing tank having an inner diameter larger than a pipe inner diameter of the water path connected to the inflow port of the liquid mixing unit.
